(19) United States
(12) Patent Application Publication
(10) Pub. No.: US 2016/0213264 A1
(43) Pub. Date: Jul. 28, 2016

(54) METHOD, SYSTEM, AND NON-TRANSITORY COMPUTER-READABLE RECORDING MEDIUM FOR MEASURING MULTIPLE BIO-SIGNALS

(71) Applicant: HUINNO, CO., LTD., Seongnam (KR)

(72) Inventors: Yeong Joon GIL, Busan (KR); Min Yong SHIN, Suwon (KR)

(21) Appl. No.: 14/864,387

(22) Filed: Sep. 24, 2015

(30) Foreign Application Priority Data
Jan. 27, 2015 (KR) 10-2015-0012855
Mar. 6, 2015 (KR) 10-2015-0031979
Mar. 6, 2015 (KR) 10-2015-0031980
Mar. 9, 2015 (KR) 10-2015-0032676
Apr. 10, 2015 (KR) 10-2015-0051015

(51) Int. Cl. A61B 5/0205; A61B 5/0402; A61B 5/0488; A61B 5/00

(52) U.S. Cl. CPC A61B 5/02055; A61B 5/0006; A61B 5/0008; A61B 5/0402; A61B 5/0488; A61B 5/725; A61B 5/0022; A61B 5/002; A61B 5/4266

(57) ABSTRACT

Methods, systems, and non-transitory computer-readable recording media are provided for measuring multiple bio-signals. A method is provided for measuring multiple bio-signals including acquiring a first bio-signal from a first device disposed at a first point on a human body. The method also includes acquiring a second bio-signal from a second device disposed at a second point on the human body. The method additionally includes quantizing the acquired first bio-signal and second bio-signal. The method further includes synchronizing the quantized first bio-signal and second bio-signal. The method also includes generating biometric information with reference to the synchronized first bio-signal and second bio-signal.

### CROSS-REFERENCE TO RELATED APPLICATION

This application claims priority from

1. Korean Patent Application No. 10-2015-0032676 filed on Mar. 9, 2015,

2. Korean Patent Application No. 10-2015-0012855 filed on Jan. 27, 2015,

3. Korean Patent Application No. 10-2015-0051015 Apr. 10, 2015,

4. Korean Patent Application No. 10-2015-0031980 Mar. 6, 2015,

5. Korean Patent Application No. 10-2015-0031979 Mar. 6, 2015, in the Korean Intellectual Property Office. The application is incorporated by reference herein.

### TECHNICAL FIELD

The present invention relates to methods, systems, and non-transitory computer-readable recording media for measuring multiple bio-signals.

### BACKGROUND

Due to rapid developments in science and technology, the quality of life of the entire human race has been improved, bringing many changes to the medical environment. It generally may take a few hours or days to have medical images, such as X-ray, computerized tomography (CT), or functional magnetic resonance imaging (fMRI) images, interpreted.

However, picture archive communication systems (PACS) have been introduced that capture medical images and transmit the captured medical images to a screen of a radiologist's monitor to allow the captured medical images to be interpreted by the radiologist. Also, ubiquitous-Healthcare (u-Healthcare)-related medical appliances that enable patients to check their own blood sugar and blood pressure anytime and anywhere have been widely distributed and are increasingly being used at homes or offices by many diabetic or hypertensive patients. Thus, in the case of hypertension, which is known to be a major cause of various diseases and has an ever-increasing prevalence, systems are needed for regularly measuring blood pressure and reporting the blood pressure in real time.

Biometric information, such as blood pressure, electrocardiogram (ECG), heart rate, body temperature information, oxygen saturation, electromyogram (EMG), sweat gland activity, volume of perspiration, and respiration rate, may be obtained based on multiple bio-signals obtained respectively from two or more contact points (not necessarily physically adjacent to each other) on the human body. Accordingly, in order to obtain biometric information, the multiple bio-signals obtained from multiple contact points of the human body must be processed and measured.

In a measurement process of the multiple bio-signals, the user may need to consciously perform an action (e.g., bringing a right-hand finger in contact with a measurement device while the measurement device is worn on the left wrist). Additionally, in order to obtain biometric information such as blood pressure, both distal ends (e.g., wrist, hand, ankle, foot, etc.) of the human body may need to be connected through wires to the measurement device.

For example, inputting two signals obtained from two distal ends of the user's body to one differential amplifier may be used to measure an ECG signal. In such a case, the user would need to consciously bring two distal ends of his/her own body in contact with input terminals of the differential amplifier or connect through wires two distal ends of his/her own body with input terminals of the differential amplifier. This effort by the user may cause inconvenience.

In addition, because the user is required to actively perform a conscious action in order to measure multiple bio-signals, in a situation where the user is in danger (e.g., unconscious), it may be difficult to measure multiple bio-signals and generate the biometric information therefrom.

Therefore, the present invention relates to a technique to obtain desired biometric information by wirelessly acquiring and synchronizing the multiple bio-signals measured respectively from two or more measurement devices physically separated from each other.

### SUMMARY

In view of the above, the present invention relates to methods, systems, and non-transitory computer-readable recording media for measuring multiple bio-signals, capable of obtaining desired biometric information by wirelessly acquiring and synchronizing the multiple bio-signals measured respectively from two or more measurement devices physically separated from each other. An exemplary method may include acquiring a first bio-signal from a first device disposed at a first point of a human body, acquiring a second bio-signal from a second device disposed at a second point of the human body, quantizing the acquired first bio-signal and second bio-signal, and synchronizing the quantized first bio-signal and second bio-signal to generate biometric information.

According to one exemplary aspect of the present invention, a method is provided for measuring multiple bio-signals, including acquiring a first bio-signal from a first device disposed at a first point of a human body, acquiring a second bio-signal from a second device disposed at a second point of the human body, quantizing the acquired first bio-signal and second bio-signal, synchronizing the quantized first bio-signal and second bio-signal, and generating biometric information with reference to the synchronized first bio-signal and second bio-signal.

According to another exemplary aspect of the present invention, a system is provided for measuring multiple bio-signals, including a bio-signal acquirer acquiring a first bio-signal from a first device disposed at a first point of a human body, acquiring a second bio-signal from a second device disposed at a second point of the human body, a bio-signal processor quantizing the acquired first bio-signal and second bio-signal, synchronizing the quantized first bio-signal and second bio-signal, and generating biometric information with reference to the synchronized first bio-signal and second bio-signal.

In addition, another exemplary method and system for implementing the present invention, and a non-transitory computer-readable recording medium storing a computer program for executing the method are further provided.

Related to the present invention, because desired biometric information can be generated by wirelessly acquiring and synchronizing the multiple bio-signals measured respectively from two or more measurement devices physically separated from each other, constantly measuring multiple bio-signals may be achieved without requiring the user to actively perform a conscious action. In addition, other systems, apparatuses, and methods for implementing the present invention may be provided.

### BRIEF DESCRIPTION OF DRAWINGS

The above and other aspects and features of the present inventive concept will become more apparent by describing in detail exemplary embodiments thereof with reference to the attached drawings, in which:

FIG. 1 is a block diagram schematically showing a configuration of the entire system according to an exemplary embodiment the present invention;

FIG. 2 exemplarily shows an internal configuration of a multiple bio-signal measurement system according to an exemplary embodiment of the present invention;

FIG. 3 is a diagram exemplarily showing a process of generating biometric information by measuring multiple bio-signals according to an exemplary embodiment of the present invention; and

FIG. 4 is a diagram exemplarily showing a process of generating electrocardiogram information by measuring multiple bio-signals according to an exemplary embodiment of the present invention.

### DETAILED DESCRIPTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Exemplary embodiments of the present invention are described in detail below with reference to the accompanying drawings. The exemplary embodiments of the present invention are sufficiently described in detail such that those skilled in the art may carry out the present invention. It should be understood that although various exemplary embodiments of the present disclosure are different from each other, they need not be mutually exclusive. For example, in regard to an embodiment, specific forms, structures, and characteristics described herein can be realized through another embodiment without departing from the spirit and scope of the present invention. Moreover, it should be understood that locations or arrangements of separate elements within the disclosed embodiments can be changed without departing from the spirit and scope of the present invention. Accordingly, detailed descriptions which will be given below are not intended to be restrictive, and the scope of the present invention is limited only by the accompanying claims and equivalents thereof. Similar reference numerals shown in the drawings denote elements performing identical or similar functions in several aspects.

Hereinafter, exemplary embodiments of the present invention will be described with reference to the accompanying drawings so that those skilled in the art to which the invention pertains may easily implement the invention.

### Configuration of the Entire System

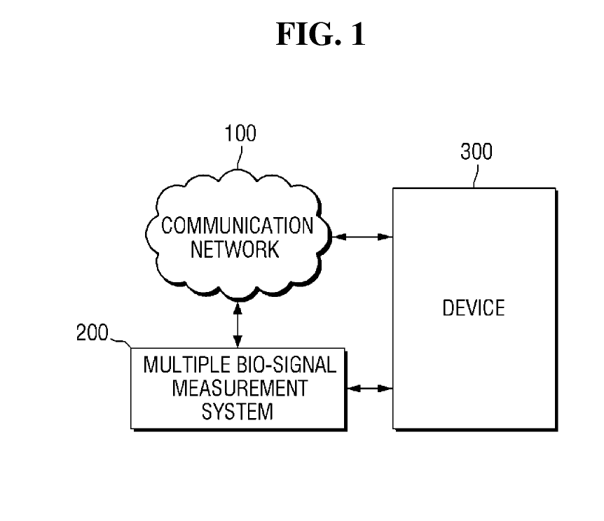

Hereinafter, a multiple bio-signal measurement system according to an exemplary embodiment will be described in detail. FIG. 1 shows a block diagram schematically showing a configuration of the entire system. As shown in FIG. 1, the entire system may include a communication network 100, a multiple bio-signal measurement system 200, and a device 300.

The communication network 100 is not necessarily limited to a particular communication method such as a wired or wireless communication method, and may be implemented as various communication networks, such as a local area network (LAN), a metropolitan area network (MAN), and/or a wide area network (WAN). Examples of the communication network 100 may include LANs, such as a Wireless-Fidelity (Wi-Fi) network, a Wi-Fi Direct network, a Long-Term Evolution (LTE) Direct network, and a Bluetooth network that are known in the art, but the present invention is in no way limited thereto. The communication network 100 may at least partially include a typical wired/wireless communication network, a typical telephone network, and/or a typical wired/wireless television communication network.

The multiple bio-signal measurement system 200 may acquire a first bio-signal from a first device disposed at a first point of a human body, may acquire a second bio-signal from a second device disposed at a second point of the human body, may quantize (e.g., may restrict to discrete values rather than to a continuous set of values) the acquired first bio-signal and second bio-signal, and may synchronize the quantized first bio-signal and second bio-signal to generate biometric information. Thus, the multiple bio-signal measurement system 200 may be configured to generate desired biometric information by wirelessly acquiring and synchronizing multiple bio-signals measured from the respective two or more measurement devices separated physically.

The function of the multiple bio-signal measurement system 200 will be described in more detail later. The multiple bio-signal measurement system 200 has been described above, but this description is exemplary. It will be apparent to those skilled in the art that at least a part of components or functions required for the multiple bio-signal measurement system 200 may be realized and/or included in the device 300.

The device 300 is a digital device that may be configured to perform communication after being connected to the multiple bio-signal measurement system 200. A digital device having a memory and a microprocessor to perform a computation may be employed as the device 300. The device 300 may be a wearable device, such as smart glass, smart watch, smart band, smart ring, and/or smart necklace, or a device such as a smart phone, smart pad, desktop computer, laptop computer, workstation, PDA, web pad, and/or mobile phone. The device 300 may include a sensor (e.g., a contact electrode, an image photographing device, etc.) for obtaining multiple bio-signals from the human body, and a display for providing biometric information to the user.

Further, the device 300 may include an application program configured to perform a function related to the present invention. This application may be present in the form of a program in the device 300. The nature of this program may be generally similar to a bio-signal acquirer 210, a bio-signal processor 220, a communication processor 230, and/or a controller 240 of the multiple bio-signal measurement system 200 as will be described later. The acquirer, bio-signal processor, communication processor, controller, any module, and/or any unit may be operated by a master controller having a memory and a processor. At least a part of the application may be replaced by a hardware device or firmware device that can perform substantially the same or equivalent function as needed.

### Configuration of the Multiple Bio-Signal Measurement System

Hereinafter, the function of each component and an internal configuration of the multiple bio-signal measurement system 200 performing a function related to the present invention will be described.

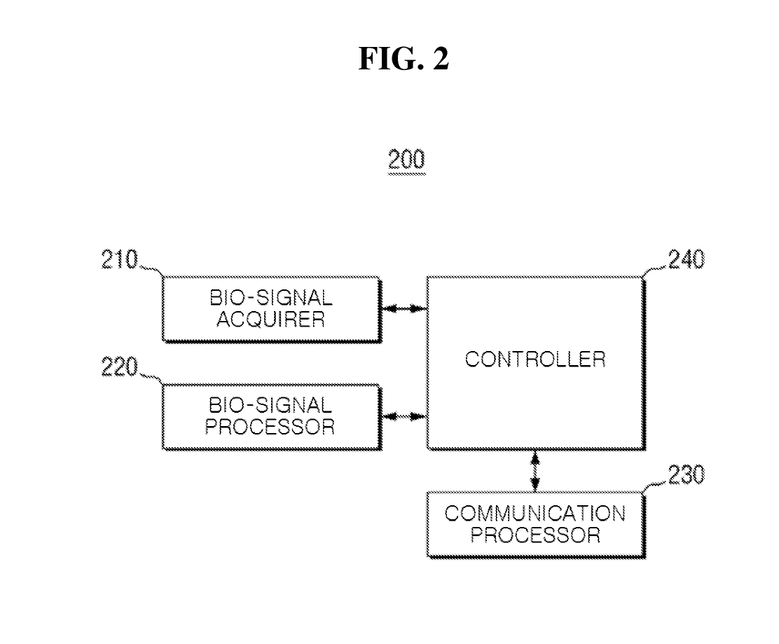

Referring to FIG. 2, the multiple bio-signal measurement system 200 may include a bio-signal acquirer 210, a bio-signal processor 220, a communication processor 230, and a controller 240. According to an exemplary embodiment, at least a part of the bio-signal acquirer 210, the bio-signal processor 220, the communication processor 230, and/or the controller 240 may be programs communicating with an external system (not shown). These programs may be included in the multiple bio-signal measurement system 200 in the form of an operating system, an application program, and other programs. These programs may physically be stored in various storage devices known in the art. Further, these programs may be stored in a remote storage device that can communicate with the multiple bio-signal measurement system 200. These programs may be configured to perform particular tasks, which will be described later, or include a routine, a sub-routine, a program, an object, a component, a data structure, etc. that perform particular abstract data types, but the present invention is not limited thereto.

First, the bio-signal acquirer 210 may be configured to acquire a first bio-signal from a first device disposed at a first point of the human body, and may be configured to acquire a second bio-signal from a second device disposed at a second point of the human body. A contact state between a first point of the human body and a first device (e.g., a state in which the first device can acquire a bio-signal from the first point of the human body even though the first device and the first point are not physically in contact with each other) and a contact state between a second point of the human body and a second device can be constantly and/or continuously maintained. Accordingly, even if the user does not actively perform a conscious action, the first device and the second device can constantly and/or continually acquire the first bio-signal and the second bio-signal, respectively.

Further, the first bio-signal and/or the second bio-signal may be acquired through a wireless communication network. For example, the first bio-signal and/or the second bio-signal may be transmitted through a known wireless local area network such as Wi-Fi, Wi-Fi Direct, LTE Direct, and/or Bluetooth. Meanwhile, as will be described later, the wireless transmission of the first bio-signal and the second bio-signal may be performed after quantization is performed on the bio-signals.

Next, the bio-signal processor 220 may be configured to quantize the first bio-signal and the second bio-signal that are acquired as described above. The bio-signal processor 220 may perform quantization on the first bio-signal and the second bio-signal acquired as analog signals from the human body, and may convert these bio-signals into digital signals, thereby reducing the amount of data to be stored or processed subsequently. The bio-signal processor 220 may be configured to synchronize the first bio-signal and the second bio-signal that are quantized as described above, and may be configured to generate desired biometric information with reference to the synchronized bio-signals.

According to an exemplary embodiment, biometric information may include information regarding electrocardiogram (ECG), heart rate, body temperature information, blood pressure, oxygen saturation, electromyogram (EMG), sweat gland activity, respiration rate, etc. However, the biometric information that may be generated in the present invention is not limited to those listed above, and other biometric information may be generated according to types of multiple bio-signals acquired through the first device and the second device.

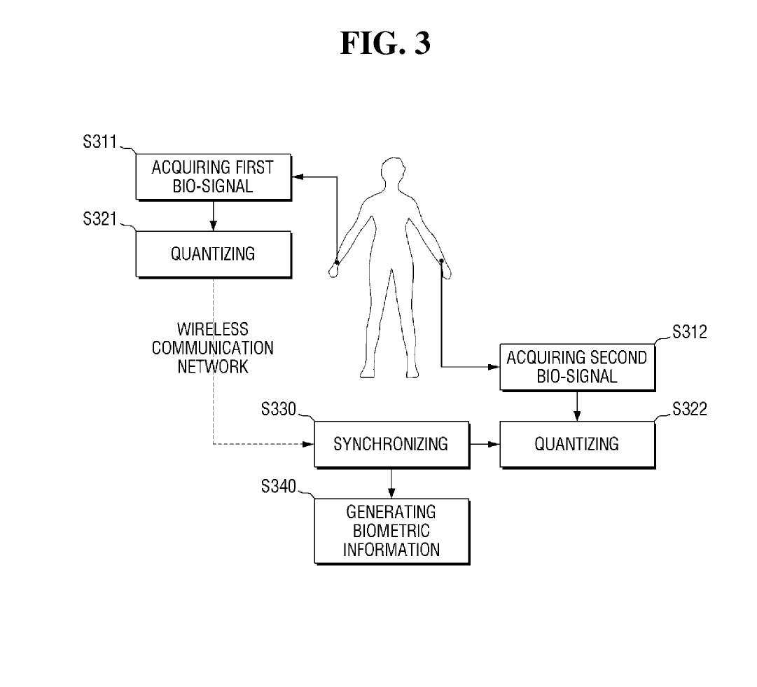

Referring to FIG. 3, the multiple bio-signal measurement system 200 may acquire a first bio-signal from a first device disposed on the user's left wrist (step S311), may acquire a second bio-signal from a second device disposed on the user's right wrist (step S312), and may perform quantization on the first bio-signal and the second bio-signal that have been acquired as described above (steps S321 and S322). Subsequently, the multiple bio-signal measurement system 200 may synchronize the first bio-signal and the second bio-signal that have been converted into digital signals according to the quantization (step S330), and generate desired biometric information with reference to the first bio-signal and the second bio-signal that have been synchronized as described above (step S340).

Meanwhile, in the case of biometric information such as electrocardiogram (ECG) information, in order to generate the biometric information, it may be necessary to process multiple bio-signals in the form of analog signals. The bio-signal processor 220 may thus be configured to perform subsequent processing required to generate bio-signals by converting the first bio-signal and the second bio-signal, which have been converted into digital signals according to the quantization, into analog signals again. The bio-signal processor 220 may convert the first bio-signal and the second bio-signal into analog signals by performing digital-to-analog conversion (DAC) on the above-quantized first bio-signal and second bio-signal, and may generate desired biometric information by performing amplification and band-pass filtering on the first bio-signal and the second bio-signal that have been converted into analog signals.

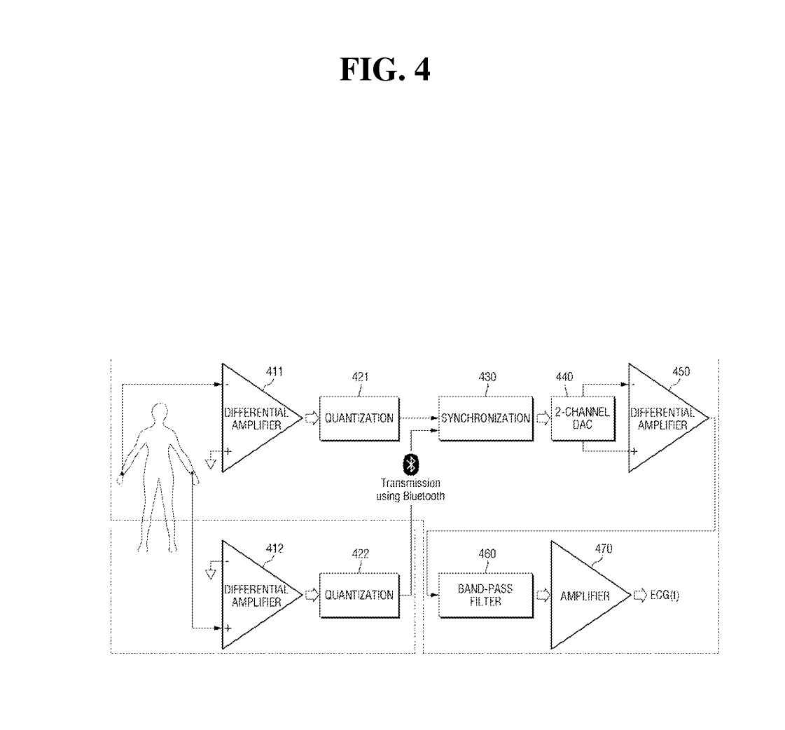

Referring to FIG. 4, the multiple bio-signal measurement system 200 may synchronize the first bio-signal and second bio-signal that have been quantized after being acquired from the first device and the second device disposed on the user's left wrist and right wrist, respectively. Subsequently, referring to FIG. 4, the multiple bio-signal measurement system 200 may perform digital-to-analog conversion on the synchronized first bio-signal and second bio-signal to convert both the first bio-signal and the second bio-signal into analog signals. The first bio-signal and the second bio-signal after conversion are inputted to a differential amplifier 450, and band-pass filtering 460 and amplification 470 are further performed on output signals from the differential amplifier 450, thereby generating electrocardiogram (ECG) information, e.g., a complete ECG signal ECG(t). The communication processor 230 may be configured to allow the multiple bio-signal measurement system 200 to communicate with an external device.

Finally, the controller 240 may be configured to control the flow of data between the bio-signal acquirer, the bio-signal processor 220, and the communication processor 230. The controller 240 controls such that each of the bio-signal acquirer 210, the bio-signal processor 220, and the communication processor 230 performs a specific function by the controller 240 controlling the flow of data from the outside of the system or between the components of the multiple bio-signal measurement system 200.

Exemplary embodiments of the present invention may be implemented in the form of program instructions that can be performed by various computing devices to be thereby recorded in a non-transitory computer-readable recording medium. The non-transitory computer-readable recording medium may include program instructions, data files, and data structures, alone or in a combination thereof. The program instructions recorded in the non-transitory computer-readable recording medium may be specially designed and configured for the present invention, or be those well-known to those skilled in the field of computer software. The non-transitory computer-readable recording medium may include magnetic media such as hard disks, floppy disks and magnetic tapes, optical media such as a Compact Disc Read-Only Memory (CD-ROM) and a Digital Versatile Disc (DVD), magneto-optical media such as floptical disks, and hardware devices such as a Read-Only Memory (ROM), a Random Access Memory (RAM) and a flash memory, which are specially configured to store and perform program instructions. Further, the program instructions include a machine language code generated by a compiler and a high-level language code executable by a computer through an interpreter and the like. The hardware devices may be configured to operate as one or more software modules to perform the operations of the present invention, and vice versa.

As described above, although the present invention has described specific matters such as concrete components, the exemplary embodiments and the drawings are provided merely to assist in a general understanding of the present invention, and the present invention is not limited to the exemplary embodiments. Various modifications and changes can be made from the description by those skilled in the art.

Accordingly, the spirit and scope of the present invention should not be limited or determined by the above-described exemplary embodiments, and it should be noted that not only the claims which will be described below but also their equivalents fall within the spirit and scope of the present invention.

What is claimed is:

1. A method for measuring multiple bio-signals, comprising:
acquiring a first bio-signal from a first device disposed at a first point on a human body, and acquiring a second bio-signal from a second device disposed at a second point on the human body;
quantizing the acquired first bio-signal and second bio-signal; and
synchronizing the quantized first bio-signal and second bio-signal, and generating biometric information with reference to the synchronized first bio-signal and second bio-signal.

2. The method of claim 1, wherein acquiring the first bio-signal from the first device disposed at the first point and acquiring the second bio-signal from the second device disposed at the second point are performed constantly.

3. The method of claim 1, wherein at least one of the first bio-signal and the second bio-signal is acquired through a wireless communication network.

4. The method of claim 1, wherein the biometric information includes information regarding at least one of electrocardiogram (ECG), heart rate, body temperature information, blood pressure, oxygen saturation, electromyogram (EMG), sweat gland activity, volume of perspiration and respiration rate.

5. The method of claim 1, wherein generating the biometric information includes:
performing digital-to-analog conversion on the synchronized first bio-signal and second bio-signal;
performing at least one process of amplification and band-pass filtering on the digital-to-analog converted first bio-signal and second bio-signal; and
generating biometric information with reference to the processed first bio-signal and second bio-signal.

6. A non-transitory computer-readable recording medium storing a computer program for executing the method according to claim 1.

7. A system for measuring multiple bio-signals, comprising:
a bio-signal acquirer configured to acquire a first bio-signal from a first device disposed at a first point on a human body, and configured to acquire a second bio-signal from a second device disposed at a second point on the human body; and
a bio-signal processor configured to quantize the acquired first bio-signal and second bio-signal, configured to synchronize the quantized first bio-signal and second bio-signal, and configured to generate biometric information with reference to the synchronized first bio-signal and second bio-signal.

8. The method of claim 1, wherein:
acquiring the first bio-signal from the first device includes continually acquiring the first bio-signal from the first device, and
acquiring the second bio-signal from the second device includes continually acquiring the second bio-signal from the second device.

9. A method for measuring multiple bio-signals, comprising:
acquiring a first bio-signal from a first device disposed at a first point on a human body;
acquiring a second bio-signal from a second device disposed at a second point on the human body;
quantizing the acquired first bio-signal and second bio-signal;
synchronizing the quantized first bio-signal and second bio-signal;
converting the synchronized first bio-signal and second bio-signal from digital to analog;
amplifying and band-pass filtering the digital-to-analog converted first bio-signal and second bio-signal; and
generating biometric information with reference to the processed first bio-signal and second bio-signal.

10. The method of claim 9, wherein the biometric information includes information regarding at least one of electrocardiogram (ECG), heart rate, body temperature information, blood pressure, oxygen saturation, electromyogram (EMG), sweat gland activity, volume of perspiration and respiration rate.

11. A non-transitory computer-readable recording medium storing a computer program for executing the method according to claim 9.
